(19) United States
(12) Patent Application Publication
(10) Pub. No.: US 2015/0033830 A1
(43) Pub. Date: Feb. 5, 2015

(54) AUTOMATED PHASE SEPARATION AND FUEL QUALITY SENSOR

(71) Applicant: DirAction, LLC, Spring City, PA (US)

(72) Inventor: Earle David DRACK, Spring City, PA (US)

(21) Appl. No.: 14/458,816

(22) Filed: Aug. 13, 2014

Related U.S. Application Data
(63) Continuation of application No. 12/812,130, filed on Aug. 12, 2010.
(60) Provisional application No. 61/010,397, filed on Jan. 9, 2008.
(60) Provisional application No. 61/196,682, filed on Oct. 21, 2008.

(51) Int. Cl. G01N 33/22; G01M 3/00; G01N 33/18

(52) U.S. Cl. CPC G01N 33/22; G01N 33/18; G01M 3/00
USPC 73/61.44

(57) ABSTRACT

A fluid characterization sensor comprising a plurality of sensor segments is disclosed. Each segment comprises two electrodes, spaced apart so the fluid in the corresponding interval of depth for that segment is positioned between them. Complex current or impedance is measured by exciting one electrode with an AC signal, and measuring the amplitude and phase of the current in the other electrode. After automatically measuring and accounting for pre-determined gain, offset, temperature, and other parasitic influences on the raw sensor signal, the complex electrical impedance of the fluid between the electrodes is calculated from the measured phase/amplitude and/or real/imaginary components of the received electrical current signal and/or the variation of the measured response with variation in excitation frequency. Comparison of measured results with results taken using known fluids identifies fluid properties. Alternatively, measured results are compared to predicted results using forward models describing expected results for different fluids or contaminants.

### CROSS-REFERENCE TO RELATED APPLICATIONS

This utility application claims the benefit under 35 U.S.C. §120 as a continuation of national stage application Ser. No. 12/812,130 filed on Aug. 12, 2010, which claims the benefit under 35 U.S.C. §371 of PCT/US2009/030427, filed on Jan. 8, 2009, entitled AUTOMATED PHASE SEPARATION AND FUEL QUALITY SENSOR, which claims the benefit of under 35 U.S.C. §119(e) of Provisional Application Ser. No. 61/010,397, filed on Jan. 9, 2008, entitled AUTOMATED PHASE SEPARATION AND FUEL QUALITY SENSOR; and Provisional Application Ser. No. 61/196,682, filed on, Oct. 21, 2008 entitled, SYSTEM FOR FUEL QUALITY DETECTION AND NOTIFICATION; the entire disclosures of which are incorporated by reference herein.

### FIELD OF THE INVENTION

The disclosure relates to the fields of liquid level detection and fluid property measurement, and in particular level detection, leak detection, and fuel quality measurement of mixed fluids, including ethanol, gasoline, and water.

### BACKGROUND OF THE INVENTION

Liquid fuel for retail and commercial use is often stored in above-ground storage tanks (AST's) and underground storage tanks (UST's). These tanks supply dispensers from which the fuel is pumped into vehicles or other storage tanks. Over the years, instrumentation was developed to automatically monitor the level of fuel product in such tanks. Such instrumentation, often referred to as an Automatic Tank Gauge or ATG, typically includes a probe section which extends into the tank and contains level and temperature sensors for conversion of product level measurement to product volume based on known shape of tank and temperature effects. In addition to the sensor probe, electronics are used to condition the sensor signals, provide excitation if necessary, and to process the sensor data. The resulting product level information is displayed and recorded.

In addition to fuel product level measurement, many systems also contain a means for measuring the level of water residing at the bottom of the tank. The most popular means of measuring product and water level in retail fuel sales settings is by means of magnetostrictive probes. Such probes use a system of floats which slide up and down a tube which contains a magnetostrictive element. The height of the product level float (upper, less-dense float) and water level float (lower, more-dense float) is detected by means of a magnet embedded in the floats. Upon excitation of the magnetostrictive element, a signal is created which is used to determine the vertical position of the floats in the tank. This information is used to calculate the level of product and of water.

Such methods work well for “neat” liquid fuels, with fuels containing MTBE as an oxygenating additive, and for many fuels which are significantly less dense than water and which do not mix with water. Such fuel systems will, in the presence of water ingress into the storage tank, immediately separate into two layers with distinctly different, and known, densities, allowing for the design of two-float systems which will have one float positioned on the surface of the fuel product, while the second float is positioned at fuel/water interface.

Traditional magnetostrictive buoyancy float sensors do not operate properly, however, in tanks where the fuel product contains a significant percentage volume (more than a few tenths of a percent) of ethanol. In these cases, due to the miscibility of ethanol and water, the addition of small amounts of water results first in a mixture of gasoline, ethanol, and water (i.e. the water does not form a layer at the bottom, but mixes well with the ethanol-blended fuel). As more water is added, however, the gasoline/ethanol/water system reaches a point when it can no longer remain a stable mixture. Beyond that point, most of the ethanol and water will “fall out” of the mixture in a process known as “phase separation,” leaving a layer of low density gasoline on top and a layer of aqueous ethanol which has a slightly higher density than the gasoline, on the bottom. When this happens in a tank being monitored by a typical magnetostrictive probe system, the water float will not raise up to float on the aqueous ethanol layer, since the density of that layer is much less than the density of pure water for which the water float was designed. Instead, the water float may remain at the bottom of the tank, and not indicate that aqueous ethanol layer is at the bottom of the tank. This means that the phase separation event can go undetected.

Additionally, the density of the aqueous ethanol is so close to the density of the fuel that the design of a float sensor which will reliably float on the aqueous ethanol but sink in the fuel under all conditions of fuel and temperature variation is extremely problematic. This problem is made worse by the fact that the amount of water which can be absorbed in a fuel blend varies with temperature and ethanol content, such that phase separation can occur as the result of only a change in temperature.

A related problem to phase-separation detection is the monitoring of sump and dispenser basins in a fuel station environment. The current approach to this application includes magnetostrictive probes which suffer from the fact that a relatively large amount of liquid is required to achieve float “lift-off” from the bottom, hence some water leakage into the sump or basin may go undetected because a low level of water will not be enough to lift the probe. Another problem with magnetostrictive probes is their ability to discriminate between different types of fluids based on buoyancy differences are limited. Another approach, the use of conductive polymers to detect presence of hydrocarbons, suffers from the fact that it has a very non-linear response, and triggers on even minute quantities of hydrocarbons, with the result that the indication is qualitative and not quantitative. It also is difficult or impossible to test these devices, and to reset them once they have triggered. An invention which solves these problems would be useful in sump and basin applications involving any fuels, not only those which contain ethanol.

A tank gauge sensor which will be of use in the storage of ethanol-containing fluids must therefore be based on measurement of a physical property or properties which differ significantly between:

1) the vapor-filled empty “head space” above the liquid level of the fuel,
2) the ethanol-blended fuel in its pure state,
3) the fuel when contaminated by relatively small amounts of water,
4) the aqueous ethanol bottom layer that results after phase separation has occurred,
5) the “neat” fuel upper layer that results after phase separation has occurred,
6) relatively pure water as may result from condensation of water vapor inside the tank, and
7) water contaminated with electrolytic impurities, such as road salt, that may result from storm water “runoff” leakage into a tank.

Density-based sensors do not adequately discriminate between all of the phases above, therefore a fluid level sensor is needed that can properly discriminate between the different substances and phases of the substances.

### SUMMARY OF THE INVENTION

An embodiment of the present invention is a complex electrical current sensor which extends into a storage tank or other container. The sensor comprises a plurality of sensor segments, arranged vertically. Each segment comprises two electrodes, which are spaced apart such that the fluid in the corresponding interval of the tank depth for that segment is positioned between them. Complex (magnitude and phase) electrical current is measured by exciting one electrode with an AC signal at one or more known frequencies and amplitudes, and measuring the amplitude and phase of the current that is collected in the other electrode. After automatically measuring and accounting for pre-determined gain, offset, temperature, and other parasitic influences on the raw sensor signal, the complex electrical current, or the impedance of the sample fluid between the electrodes, is calculated from the measured phase/amplitude and/or real/imaginary components of the received electrical signal and/or the variation of the measured response with variation in excitation frequency.

A series of equations and/or tables are solved and/or used to assign a fluid type or types and physical phase or phases for that interval in the tank based on the measured response, the known physical properties of the possible fluids, as well as other measured, known, or assumed parameters such as temperature, pressure, etc.

By repeating this process for an array of electrode pairs, a profile of the fluid distribution over the length of the sensor is generated. That profile, combined with known position of the sensor in the tank, is used to determine the overall liquid level in the tank by determining the position of an interface between liquid and vapor phase, assuming that interface exists within the sensor boundaries. In an embodiment, the profile is also used to determine the presence of and/or level of water and/or aqueous ethanol (by determining the position of an interface between dissimilar liquids and/or the properties of the liquids between the segment electrodes), and thus provides an alert that phase separation or water ingress has occurred as well as the extent of the contamination.

In a further embodiment, in addition to or instead of using the differences between segment responses to determine boundary layer location, some or all of the segment responses may be combined to improve the precision and accuracy of the resulting measurements once it has been established that the fluid at each segment to be combined is essentially the same as the fluid in the other segments with which it is to be combined.

In a further embodiment, the complex current or complex impedance data is fit to a model comprising a plurality of complex current or complex impedance elements in various configurations, and the model solved for those element values (including the value of those elements which correspond to the fluid of interest as well as other parasitic elements). In that manner improved accuracy can be achieved as parasitic impedances can be better accounted for and their effects removed prior to the fluid identification phase as compared to a single element model or a simple parallel or series R, C, RC, or RLC model.

In a further embodiment, for each application, the height, segment number, spacing, and size of the array of segment electrodes is tailored to yield the desired vertical resolution for the level and interface location measurement. In a further embodiment, the device is oriented such that it is not orthogonal to the liquid surfaces to be measured. In such an orientation, vertical resolution is improved without sacrificing signal to noise ratio (SNR) by making segments smaller for a given width.

In a further embodiment, comparison of the individual segment data is used as a quality control check to ensure that basic assumptions about possible fluid configurations are met. In a further embodiment, adjacent segment measurements are used to interpolate and improve accuracy of interface position estimate when the interface between two fluids falls within a segment.

In addition to allowing the determination of type and phase of the fluid, the measured complex electrical current or impedance is also used to provide a useful indication of fuel quality and/or contamination. For example, relatively high current or high conductivity in the water or aqueous ethanol phase can indicate water which has electrolytic contamination as may indicate a leak that allowed storm-water run-off to enter the tank. Relatively low current or conductivity may indicate that the water present is the result of condensation. Similarly, variations in the complex current or impedance measurement can give an indication of absorbed water in the fuel even prior to phase separation, providing an opportunity to address the problem before a costly phase separation event has occurred. Complex current or impedance variations can also indicate contamination by other substances besides water, as well as the percentage of ethanol present.

A further embodiment is an automated leak detection system which includes an automated phase separation and water measurement system for ethanol blended or non-ethanol-blended fuels, or any other fluids, including a sensor of the type described herein or a different sensor for measuring water content. The sensor provides an indication of lack of water seal if water ingress has been detected. This embodiment has and advantage over the prior art in that a leak detection system which only measures product/vapor interface level cannot accurately detect a leakage event under all circumstances because leaking product can be offset by a corresponding amount of water ingress.

In another embodiment, the sensor electrode surfaces are coated with chemically resistant materials to allow for prolonged use in a fuel tank environment, and the effect of that coating on the impedance measurement is measured and compensated for. In a further embodiment, a seal is placed between the electrode segments and the electronics package for the sensor, which may include power, excitation, automatic gain ranging, frequency sweeping or hopping, data acquisition, data processing, control, and communications circuits.

In a further embodiment, the sensor described herein is integrated into the lower end of a prior art magnetostrictive buoyancy probe. This combination maintains the position accuracy and operation of the product level float (and the extensive industry infrastructure of software and hardware based on that measurement), but augments that with phase separation detection, water detection, and/or fuel quality measurements performed in the lower interval by the complex current/impedance sensor. In the case where product level drops into the range covered by the complex current/impedance sensor, it can measure that level as well by providing the vertical position of the liquid/vapor interface which defines the product level. Such a hybrid probe is also suited to be a part of the method and apparatus for the automated ethanol blend leak detection system described above.

In a further embodiment, the sensor includes a circuit to detect an electrical signal from one or both segment electrodes, properties of said electrical signal varying according to known applied signal properties and unknown fluid properties. In a further embodiment, the electrical properties detected include complex electrical current or impedance.

In a further embodiment, complex electrical current measurement consists of signal detection and signal processing to account for known signal frequency, signal amplitude, systems scale factors, gain variations, and/or offset variations to yield complex electrical current (magnitude and phase) passing through the fluid which is situated between the sensor segments.

In a further embodiment, complex electrical impedance measurement consists of signal detection and signal processing to account for known signal frequency, signal amplitude, systems scale factors, gain variations, and/or offset variations to yield complex electrical impedance (magnitude and phase) of the fluid between the sensor segments.

In a further embodiment the geometry of sensor segments is taken into account when making complex current or impedance measurements, such that the measured current or impedance, combined with known electrode geometry, are used to solve directly for electrical properties of the fluid between the electrodes.

In a further embodiment, the sensor uses a calibration scheme that includes complex current or impedance measurement of reference fluid samples, storage of those measurement results, and comparison of new measurements to reference measurements to make determinations about fluid ID or fluid characteristics.

In a further embodiment, the complex current or complex impedance measurements are performed at a single frequency. In a further embodiment, the measurements are performed at a plurality of frequencies or utilizing a frequency “sweep.”

In a further embodiment, complex current or fluid complex impedance is monitored over time and the sensor is connected to a controller that alerts an operator to changes and trends, which may indicate changes of interest to the contents of the tank or container being monitored. Such change or trend identification may be used to identify water ingress prior to phase separation occurring, since the sensor is able to detect the presence of water in a mixed state in an ethanol blended fuel even in quantities below what is necessary to cause phase separation.

In a further embodiment, the sensor is part of a system that provides input to a leak detection system to augment overall tank content level in assessing whether leakage is present.

In a further embodiment, the sensor is used to monitor a storage tank bottom for aqueous ethanol resulting from phase separation of water and ethanol from an ethanol blended fuel.

In a further embodiment, the sensor is deployed in a sump or basin to detect presence of liquid and to discriminate between water and hydrocarbons.

In a further embodiment, the sensor electrodes have a thin electrically insulating coating over sensor segment electrodes to make them less susceptible to errors caused by contamination which allows electrical leakage between electrodes. In a further embodiment, the coating is hydrophobic. In a further embodiment, the coating is a low surface energy coating such as parylene or Teflon to minimize attraction of contaminants.

In a further embodiment, the sensor includes a temperature sensor or temperature input to further refine the accuracy of the fluid identification and properties. This is done by comparing measured or provided temperature to calibration temperature and making known adjustments to physical properties which are temperature-dependent or by incorporating temperature into fluid property calculations based on excitation signal, electrode geometry and measured electrical response.

In a further embodiment, the sensor uses a lumped electrical circuit model, based on known sensor characteristics, to represent the sensor segment system, and solves a series of equations to calculate parasitic electrical elements in the system, data for equation solutions coming from a series of measurements at varying frequencies. These parasitic elements, once identified, can be used to improve the accuracy and precision of the fluid measurements by taking into account the effects of the parasitic elements.

In a further embodiment, the sensor uses digital signal processing (DSP) to calculate the magnitude and phase of the complex current or complex impedance for the fluid sample between segment electrodes, eliminating errors associated with circuits which employ analog peak detection and analog phase detection.

In a further embodiment, the sensor uses data processing to remove the influences of parasitic electrical elements and thus make the fluid property measurement more accurate. In a further embodiment, the sensor uses automatic gain and amplitude control to increase the dynamic range of the measurement system, allowing it to accurately measure electrical parameters of fluids with a very wide range of complex electrical currents or impedances (e.g. air or vapor with low current/high impedance vs. salt water with high current/low impedance). In a further embodiment automatic gain control and excitation signal level control operate by monitoring magnitude of the received complex current signal and optimize both excitation amplitude and input gain to achieve maximum input signal-to-noise ratio without saturation of any stage of the input or output signal path. In a further embodiment, the automatic gain control monitors sensor data for indication of saturation in the input or output signal path and reduces gain and/or excitation signal level if saturation is detected.

In a further embodiment the sensor is integrated into a magnetostrictive product level probe.

In a further embodiment, the sensor is manufactured with carefully controlled dimensions and electrode size and spacing, and utilizes a circuit designed for accuracy and repeatability, such that a single calibration or set of data processing equations is sufficient for use in processing data from a fleet of many similar sensors with sufficient accuracy. Such a manufacturing scheme reduces individual sensor cost and lead time since each sensor does not need to be individually calibrated.

In a further embodiment the sensor segments or segment arrays are fabricated on the same PCB as the electrical circuit.

In a further embodiment, the sensor is part of a system that maps the complex current or impedance measurements and associated fluid identification or characteristics to the known depth of the sensor array segment (if using a plurality of segments) to which it corresponds, thus creating a vertical profile of fluid characteristics in the tank or container.

In a further embodiment, the sensor uses information from adjacent segment measurements to determine whether a fluid transition interface has occurred between adjacent segments or within a segment. In a further embodiment, the sensor uses information from adjacent segments to calculate where in a segment a fluid transition occurs, based on complex current or impedance from the segment above, complex current or impedance from the segment below, relative segment geometry, and complex current or complex impedance measured in the segment.

In a further embodiment the sensor has segments of varying dimensions, allowing for more vertical resolution at some depths versus others for a given overall sensor size. In a further embodiment, the sensor has redundant sensor segments at some or all depths to allow for error detection and correction. In a further embodiment the sensor is adapted to allow liquid to circulate freely within the sensor between segment electrodes, and for liquid to drain out when sensor is removed from tank. To accomplish this, the sensor may, for example, include holes, slots, or a combination thereof, in the outer housing, if any.

In a further embodiment, the sensor has a seal between the electronics section and the sensor section, where the seal comprises a material resistant to the fuels in which the sensor will be placed. In a further embodiment, the sensor includes an intrinsically safe circuit design for use in hazardous locations.

In a further embodiment, the sensor has a seal that adheres directly to a circuit board as well as an outer housing, allowing the sensor to be made inexpensively using PCB traces passing through the PCB and thus through the seal to connect the sensor in the fuel or other fluid area to the electronics active area. In a further embodiment, the seal comprises a feedthrough bulkhead which utilizes a glass-to-metal or other seal to isolate the sensor in the fuel or other fluid from the electronics.

In a further embodiment, the sensor transmits data to display and/or recording devices for inspection. In a further embodiment, the sensor transmits data to a comprehensive fuel management system.

In a further embodiment, the sensor is part of a system that detects error conditions and system malfunction by comparing calculated fluid identification over a vertical profile to possible profiles based on relative densities (e.g. water cannot float on gasoline).

In a further embodiment, the sensor is part of a system that uses complex electrical current or impedance to determine fuel quality characteristics, including but not limited to fuel type, ethanol content, water content, and presence of adulterating substances or contaminants.

In a further embodiment, the sensor is part of a system that uses complex current or complex impedance to determine electrical properties of a fluid or fluids in a container, or to infer fluid type or characteristics of the fluid or fluids in the container.

In a further embodiment, a leak detection system for ethanol and ethanol-blended fuel storage tanks monitors the tank for the presence of water as well as aqueous ethanol resulting from phase separation, ethanol, other fuels, or other fluids which may or may not be detected by an buoyancy-based ATG water float, but presence of which may indicate ingress, phase separation, and/or condensation of water or other liquid into the tank. Such ingress may mask a corresponding amount of leakage of product out of the tank, rendering leak detection unreliable if it is based only on overall level of liquid in the tank. The system uses any one or more of the fluid's electrical properties, density, or optical properties to monitor for the presence of fluid ingress or condensation.

In a further embodiment, a leak detection system monitors a container for leaks, such leak detection system incorporating information about water or other liquid ingress in addition to simply monitoring level of liquid in the container. Such a water detection and measurement may be done via any water sensing methods including electrical properties, buoyancy, optical methods, or other methods. By measuring water and incorporating that information into the leak detection algorithm, certain classes of leaks that may not be evident via total liquid level monitoring may thus be exposed.

In a further embodiment, the sensor provides data to a leak detection algorithm which uses evidence of potential fluid ingress or condensation. The leak detection algorithm includes flagging situations where fluid ingress is suspected and alerting operator that leak detection is not valid until ingress has been identified and rectified and water, aqueous ethanol, or other undesired fluids removed.

In a further embodiment, some or all of the sensor segment electrodes are coupled to each other using single or combinations of lumped or distributed electrical elements such as resistors, capacitors, inductors, and/or diodes, presenting a single measurement port for multiple segments. Frequency sweep of complex current or complex impedance at this port will yield information about the fluid properties for all segments. In a further embodiment, additional measurements at different points are made to further refine the accuracy and precision of the fluid properties for each segment. Since the electrical characteristics of elements coupling segments together are known, the fluid properties at each segment can be derived through an inversion process, involving optimization of fit between modeled response of lumped element representation of the sensor array and actual measurements at multiple frequencies. Numerous suitable algorithms are known to those skilled in the art, including but not limited to: Nelder—Mead Simplex Method (Reference: Lagarias, J. C., J. A. Reeds, M. H. Wright, and P. E. Wright, “Convergence Properties of the Nelder-Mead Simplex Method in Low Dimensions,” SIAM Journal of Optimization, Vol. 9 Number 1, pp. 112-147, 1998.) or Gauss-Newton algorithm (Fletcher, Roger (1987), Practical methods of optimization (2nd ed.), New York: John Wiley & Sons, p. 113) In a further embodiment, constraints are used when inverting the data to calculate the complex currents corresponding to each segment, such that known relationships of fluid locations (e.g. water cannot float on top of gasoline) to reduce the number of solutions and thus converge on the correct solution faster and more reliably in the presence of electrical noise.

In a further embodiment, a coupled-segment version of the sensor is deployed, and complex or scalar voltage is measured at one or more segment electrodes as a means for determining the properties and/or characteristics of the fluid situated between segment electrodes.

### DESCRIPTION OF THE DRAWINGS

FIG. 1 is a diagram of an embodiment of the sensor deployed in an underground storage tank, demonstrating the position at the bottom of the tank in order to detect a phase separation event;

FIG. 2 is a block diagram of an embodiment of the invention;

FIG. 3 is a drawing of a printed circuit board layout for an embodiment of the invention;

FIG. 4 is a flow chart describing one embodiment of the leak detection invention;

FIG. 5 is a drawing of an embodiment of the sensor which uses coupled segments; and

FIG. 6 is a drawing of an embodiment which uses coupled segments and complex voltage monitoring.

FIG. 7 is a flow chart describing the inversion process for data obtained from the embodiment of the sensor with coupled sensor segments.

FIG. 8a shows modeling results in magnitude of the inversion process described in FIG. 7, using resistors as the segment coupling elements.

FIG. 8b shows modeling results in phase of the inversion process described in FIG. 7, using resistors as the segment coupling elements.

### DESCRIPTION OF THE PREFERRED EMBODIMENT OF THE INVENTION

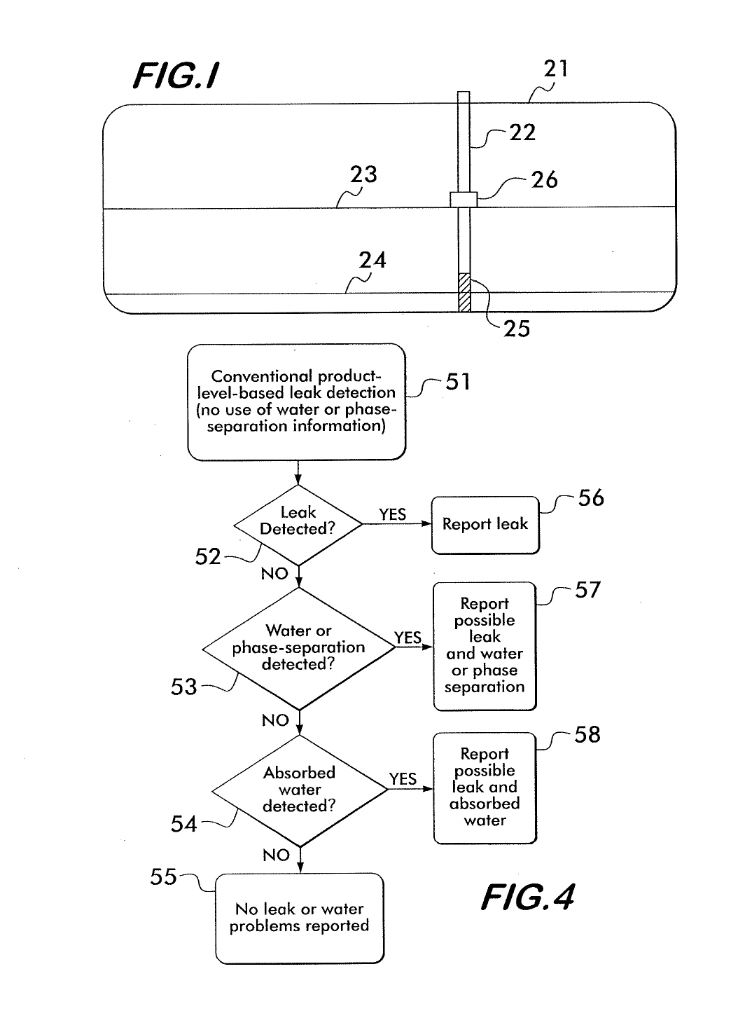

In FIG. 1, a magnetostrictive-probe-based Automated Tank Gauge or ATG (22) is deployed in storage tank (21) which contains a liquid product up to a certain level (23). The product float (26) floats on the product surface and provides an indication of product level to the ATG. An embodiment of the present invention is represented as a sensor (25) deployed at the bottom of the ATG probe. Wiring passing through the ATG powers this type of embodiment and allows for data from the sensor to be passed to the ATG control panel.

If the liquid stored in the tank is a ethanol-blended fuel, and if water is present such that phase separation has occurred, a level of aqueous ethanol (24) will form at the bottom of the tank. If such an aqueous ethanol layer covers the active region of sensor (25) then the sensor will detect the aqueous ethanol and report the problem to the control panel.

Even in cases where phase separation is not present, the sensor (25) can monitor the contents of the tank and provide an indication of changes in the fluid properties, including the presence of absorbed water prior to phase separation.

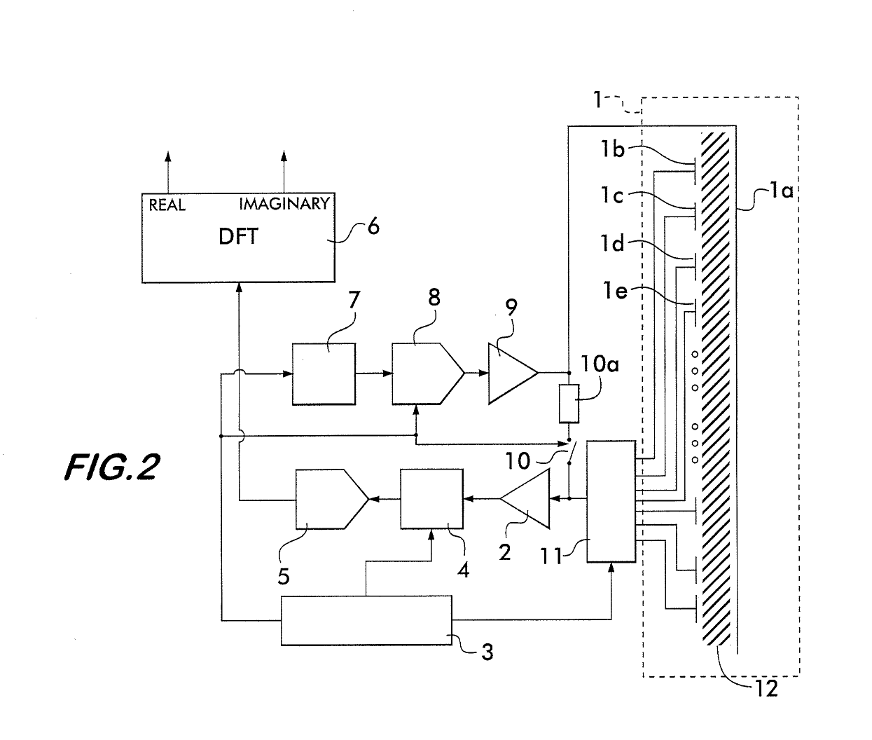

In FIG. 2, a sensor (1) is deployed such that the fluid of interest (12) is free to occupy the volume between two or more electrodes. In the embodiment shown, there is one common electrode (1a) and one or more segment electrodes (1b, 1c, 1d, 1e, 1f), each of which segment electrode occupies a unique vertical location in the container. The number and size of the segment electrodes will determine to a large extent the vertical resolution of the measurement.

Under control of a microprocessor-based timing and control circuit (3), the system generates a typically sinusoidal excitation signal via a direct-digital-synthesis circuit (7), a digital-to-analog converter (8), and a filter/driver (9). This excitation signal is impressed on a common electrode (1a) which spans the entire sensor length and, in conjunction with each of the segment electrodes (1b-1f), defines the unique electrode pairs between which the fluid of interest (12) exists.

The impressed excitation signal causes a current to flow through the fluid of interest. The characteristics of this current (its amplitude and its electrical phase relative to the excitation signal) are a function of the fluid's electrical properties (conductivity, dielectric constant, permeability), while the frequency of the current is the same as the frequency of the excitation signal.

As such, precision measurement of the current amplitude and phase can be achieved by utilizing the fact that its frequency is known to be the same as the excitation frequency and the amplitude and phase of the excitation signal are also known. Once measured, the amplitude and phase of the current from each segment yields information about the fluid properties, and allows for identification of the fluid characteristics, at the height corresponding to that segment.

A switch/multiplexer (11) may be controlled such that each segment is in turn selected and isolated from the other segments and routed to the input transimpedance amplifier (2) while the other segments may be connected together and/or connected to a common, low impedance point in order to reduce parasitic coupling of the signals. The transimpedance amplifier (2) converts the current from the selected segment to a voltage, which is run through an anti-aliasing low-pass filter (4) and digitized via an analog-to-digital-converter (5).

Once the measured current has been digitized, a digital-signal-processor (DSP) (6) is used to calculate the real and imaginary components of this signal by utilizing the known frequency and phase of the excitation signal. The frequency, amplitude, and phase of the excitation signal are set by the system, but an additional step of measuring these parameters via a switch (10) and calibration impedance (10a) to route the excitation signal to the amplifier without interacting with the fluid of interest is provided for to improve accuracy and precision, and thus allow for a more sensitive system in terms of response to changes in fluid parameters.

This process may be repeated for a plurality of different frequencies, and the additional resulting data used to refine the estimate of the fluid properties for a particular segment. Multi-frequency complex current or impedance data may also be used to solve for a particular model of lumped fluid impedances, resulting in a robust inversion of the measurement data.

The process of digitizing the current sensor output and using DSP to determine the real and imaginary current or impedance components results in enhanced accuracy over systems which use an analog method such as peak detection.

The measurement described above may be repeated for a plurality of electrode segments corresponding to different positions in the fluid container, and in this way a profile of fluid properties can be developed which describes the spatial distribution of different fluids or fluid properties within the container. When measuring one segment, other segments may be grounded and/or connected together via a switch/multiplexer in order to reduce the effect of parasitic coupling.

Complex current measurement data can be obtained in this way for a variety of fluid types and fluid mixtures. Once a library of such current measurements have been obtained, they can be used to compare new data from unknown fluids such that the unknown fluids or fluid properties can be identified via that comparison. Alternatively, an analytical model can be produced, based on electrode geometry and known fluid properties, such that the complex current measurements can be used to predict the unknown fluid type and/or properties without using stored reference measurements from known fluids.

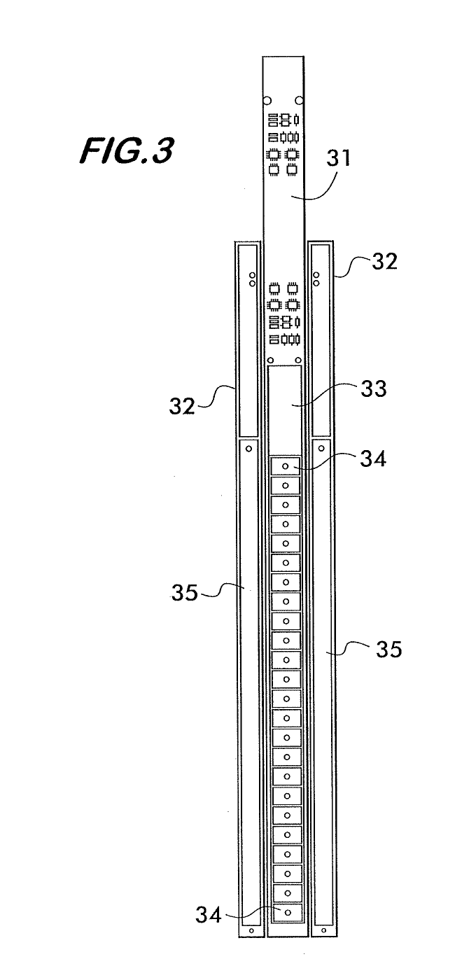

FIG. 3 shows a PCB layout line drawing of the silkscreen and top metal layers of a PC board set which is used to implement an embodiment of the invention. The main board (31) has the signal conditioning, control, excitation, signal processing, communications, and other electronics at the upper end. The PCB has double sided metallization and components on each side. On the lower end of the same board (34) are the sensor segment electrodes fabricated as double-sided copper pads connected by a plated through hole.

Between the upper electronics section of (31) and the sensor section (32) is an area that is left open (33). This area may be used to accommodate a sealing material that adheres to the PCB and to the inner surface of a mounting pipe or other structure.

The two side boards (32) are designed primarily to serve as the common electrode for the sensor segments, and the metallization (35) is configured to allow the side boards to be placed in parallel with the main board, one on either side, with the common electrodes (35) facing the double-sided sensor segments (34) the fluid of interest will be situated between the electrode faces.

In FIG. 4 the flow chart refers to an embodiment of the leak detection invention incorporating phase-separation detection and/or water detection and/or fuel quality measurement. This embodiment would be preferred in a case where product-level-based or other leak detection means are already in place and are to be augmented by the addition of phase-separation detection and or water/fuel quality measurement. If such a product-level-based detection scheme (51) detects a leak at stage (52), the leak is reported as usual (56). If no leak is detected by the product-level detection methods, the next stage (53) checks to see if the phase-separation and/or water sensor has detected phase-separation and/or water. If so, that fact is reported at (57). If not, and additional check at stage (54) uses the sensor to determine if water has been absorbed by the product. If so, that fact is reported at (58), and if not then leak testing has passed (55),

For leak detection with fuels that are not miscible with water the check for absorbed water may be skipped. For leak detection with fuels that are not susceptible to phase separation, the phase separation check may be skipped. Any water measurement method or phase separation measurement method may be used as an input to the leak detection algorithm.

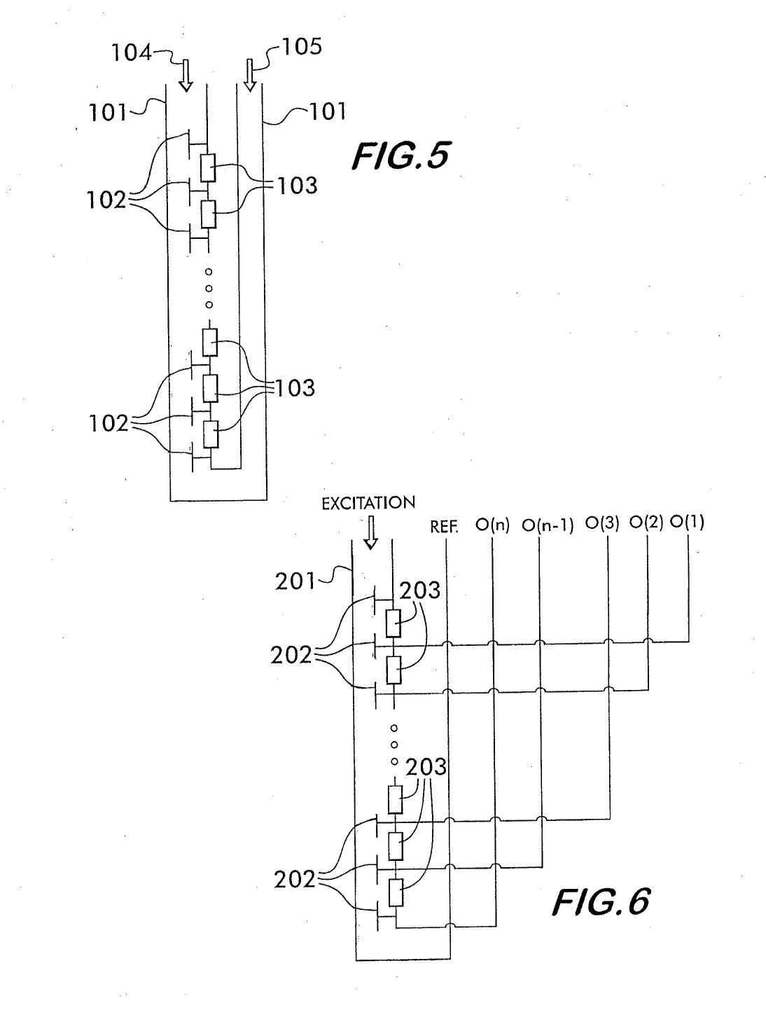

FIG. 5 shows an embodiment of the sensor which has the individual segments coupled together by discrete electrical elements. In this embodiment the hardware needed for excitation and measurement is essentially the same as shown in FIG. 2. The drawing shows the sensor array on n segments electrodes (102) and a single common electrode (101). In this case, instead of being electrically isolated, the segment electrodes are coupled by discrete elements (103) in series with the segments. Such elements can be resistors, capacitors, inductors, diodes, or combinations of those devices.

This configuration allows for information about all segments to be gathered by a single measurement at port (104) or (105), or multiple measurements at (104) and (105). Such measurements are substantially the same as those described earlier for the preferred embodiment shown in FIG. 2.

This embodiment has the advantage of requiring fewer connections between the electronics portion and the segment portion in cases where there is more than one segment, leading to reduced cost and complexity, as well as increased reliability.

For each measurement at port (104) or (105), the measurement may be made with the unmeasured port electrically shorted and/or electrically open. For each measurement at port (104) or (105), the measurement may be made with the unmeasured port electrically shorted and/or electrically open.

In this embodiment, intermediate nodes between segments may be routed to the switch/multiplexor (11) for use in calibrating and characterizing the parasitic impedances for the segments, allowing for more accurate and precise measurements.

In FIG. 6, the coupled sensor segment concept is retained as in FIG. 5. The common electrode is (201) and the segment electrodes are (202) In this case, though, the measurement technique is voltage picked off from one or more of the segments. By making a voltage measurement (complex or magnitude only) of O₁-Oₙ vs. ref, the characteristics of the fluid corresponding to each segment can be determined by solving for the corresponding electrical characteristics or by comparing the response to a library of known responses. In such a measurement, the characteristics (amplitude, phase, frequency) of the excitation signal are known or set or measured.

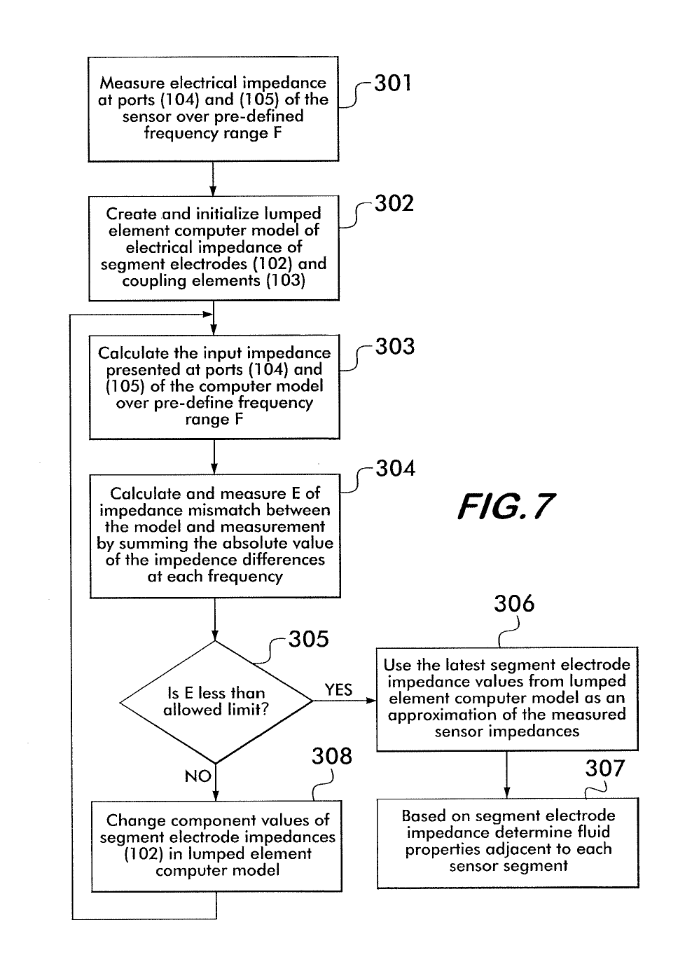

The flowchart in FIG. 7 illustrates the steps involved in inversion of the measurement results obtained from sensor embodiment as in FIG. 5. The measurement is acquired from the sensor in step (301) over pre-determined frequency range F with sufficient number of frequencies. More frequencies increase the accuracy of inversion and allows for unique solution of larger number of segments.

The lumped element computer model following the sensor topology shown in FIG. 5 is initialized in step (302) with arbitrary starting point, for example an equivalent of all sensor segments immersed in fuel. The initial conditions have impact on the number of iterations needed to achieve accurate solution and consequently the computing time.

The input impedance at the ports of the computer model are calculated in step (303) and compared to the measurement in step (304), where a measure of the mismatch is calculated. If that mismatch is smaller than allowed (305) then the inversion process is completed and the segment impedances from the latest, best fitting computer model are assumed to approximate the real sensor. If match is not accurate enough then segment electrode impedances in the computer model are changed and process continues with step (303), iteratively, until sufficient match is accomplished.

The speed of the convergence can be increased by taking into account the history of the calculations to determine the direction of the steepest slope leading to minimum of measure E.

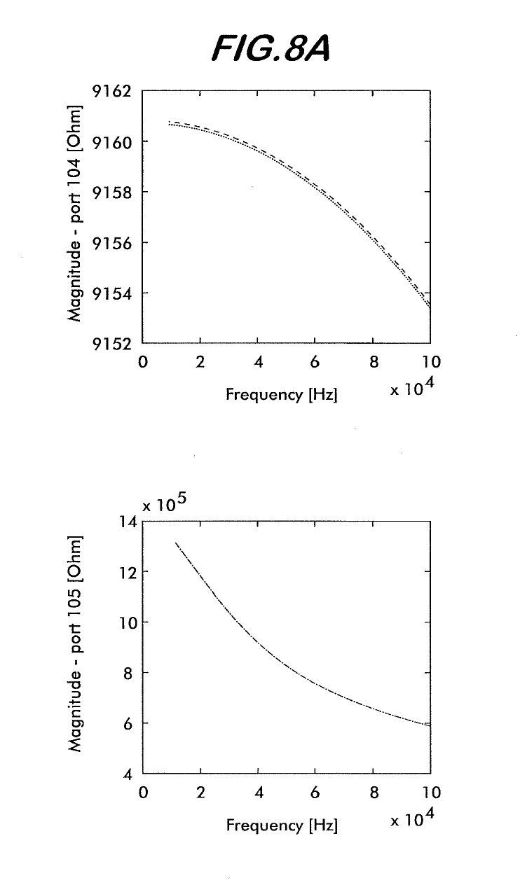

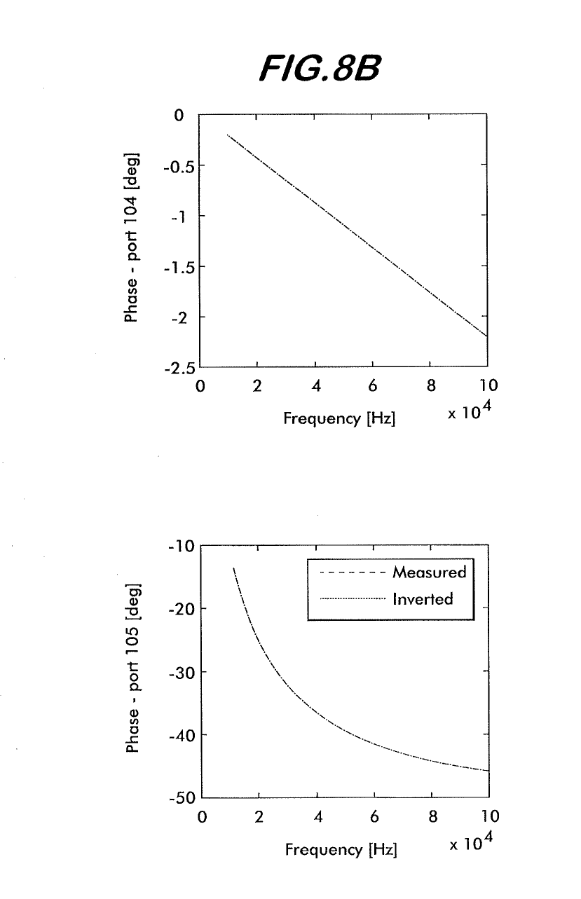

FIGS. 8a and 8b show results of modeling of the inversion process described in FIG. 7 in magnitude and phase, respectively. After some iterations the port impedances of the “measured” and “inverted” model matched to the point that the phases and magnitudes are overlapping each other. The quality of the match also depends on the measurement noise and accuracy.

### Prototypes

Prototype systems were constructed which used segment dimensions of approximately 0.25″ H×0.5″ W. Twenty two copper electrode segments, spanning approximately 6″, were constructed on both sides (connected by a played-through hole) of a main circuit board using standard Printed Circuit Board (PCB) manufacturing techniques, with the measurement electronics located on the same PCB as the electrode segments. A common electrode was configured as two strips of approximately 0.25″×6″ copper-clad PCB arranged facing each side of the main PCB. These two strips were connected electrically and served as the single common electrode (1a). The entire set of boards was contained in a pipe housing with slots in the sensor area to allow fluid to flow around the electrodes, and with a barrier between the sensor segment electrode section and the electronics section above.

With such a configuration, only a single PCB with active components is needed even when 22 or more segments are embodied. Such a solution is much less expensive to manufacture, calibrate, operate and maintain than a solution which uses active components and a separate PCB or other electronics module for each segment or spatial measurement implemented. Additional reduction in cost and complexity was achieved using integrated circuits such as the AD5933 impedance analyzer chip and a PIC microcontroller.

The prototype system PCB's included area sufficient to provide an adhesion surface for a seal between the sensor segment electrode area to be immersed in the fluid of interest and the electronic components on the main PCB. This seal can be implemented with Stycast or other materials which adhere to both the PCB and the inner surface of the pipe housing and are resistant to chemical attack by the fluids to be encountered.

The prototype system collected data primarily over a range of 10 KHz to 100 KHZ, although it is capable of extending that range to 1 KHz to 1 MHz. The entire system can be implemented without ever calculating or measuring parameters such as capacitance, dielectric constant, resistance, resistivity, etc. All that is required is the measurement of the current at each segment electrode, and either comparison of the measured value with similar measurements using known fluids or comparison with predicted current values for fluids of interest.

Alternatively, the data is displayed in any number of ways, including but not limited to complex impedances. The data can also be used to solve for values of a lumped electrical element model, such as a parallel RC and series C, representing an electrode segment with fluid between the electrodes and a thin layer of protective coating over the electrodes.

When complex impedance values were calculated, typical values for magnitude and phase with the prototype configuration were (100 KHz, single electrode segment):

### Air: 5.9 MOhm, 87 deg.

Aqueous ethanol: 27 KOhm, 15 deg.

### Distilled Water: 65 KOhm, 55 deg.

### Tap Water: 4 KOhm, 18 deg.

E10 (˜5-10% ethanol+Gasoline): 2.14 MOhm, 88 deg.

### E85: 179 KOhm, 40 deg.

Gasoline (no ethanol): 2.61 MOhm, 88 deg.

What is claimed is:

1. A method of detecting leaks in a container holding a fluid other than water, said method comprising:
measuring of one or more physical, optical or electrical parameters of the fluid;
detecting the presence of water, or a change in amount of water in said container by said measurement;
augmenting a fluid-level-based, statistical-reconciliation-based, or other leak detection method with said water detection based on a physical, optical or electrical parameter of the fluid and
noting the presence of a container leak if water is detected in the container, or if the amount of water detected in the container has changed since a previous measurement.

2. The method of claim 1 wherein the detected water is disposed in a layer at the bottom of the container, or is disposed at the top of the contained fluid if the contained fluid is more dense than water.

3. The method of claim 1 wherein the detected water is disposed in an absorbed state in an ethanol blended fuel or other fluid capable of absorbing water.

4. The method of claim 1 wherein the detected water is both absorbed in the contained fluid and is disposed substantially in a layer at the top or bottom of the contained fluid.

5. The method of claim 1 wherein some or all of the detected water exists as an aqueous ethanol layer at the bottom of a container holding an ethanol blended fuel in response to a phase separation event.

6. The method of claim 1 wherein water detection measurements are made at a plurality of depths in the container.

7. The method of claim 1 wherein said water detection measurements are made with a plurality of sensors.

8. The method of claim 7, wherein said plurality of sensors includes a plurality of types of sensor.

9. The method of claim 1 wherein complex electrical impedance of the fluid is used to determine presence of water in the container.

10. The method of claim 1 wherein complex electrical impedance of the fluid is used to determine the amount of water in the container.

11. The method of claim 1 further comprising:
measuring the temperature of the fluid and wherein an amount of measured absorbed water is used in conjunction with said temperature and the fluid type to predict propensity for ethanol-blended fuel phase separation to occur, risk of ethanol-blended fuel phase separation occurring, or conditions under which ethanol-blended phase separation would occur.
